(19) United States
(12) Patent Application Publication
(10) Pub. No.: US 2020/0077967 A1
(43) Pub. Date: Mar. 12, 2020

(54) RADIOGRAPHIC APPARATUS

(71) Applicant: SHIMADZU CORPORATION, Kyoto-shi (JP)

(72) Inventors: Hiroshi OKUMURA, Kyoto-shi (JP); Tatsuya Araki, Kyoto-shi (JP)

(73) Assignee: SHIMADZU CORPORATION, Kyoto-shi (JP)

(21) Appl. No.: 16/122,961

(22) Filed: Sep. 6, 2018

(51) Int. Cl. A61B 6/00; A61B 6/08; G21K 5/02; G21K 1/02

(52) U.S. Cl. CPC A61B 6/4035; A61B 6/08; G21K 5/02; H01J 35/025; A61B 6/4208; A61B 6/56; G21K 1/02

(57) ABSTRACT

A radiation imaging apparatus discriminates a light exposure field of visible light regardless of the color of the clothes (gown) of a subject. The apparatus for the X-ray imaging apparatus has an adjustable exposure field and a color sensor mounted to the underside of a collimator detects a color of the clothes (gown) worn by the subject on the table. Each light amount of a first LED that emits a red light, a second LED that emits a green light and a third LED that emits a blue light and a fourth LED that emits a white light is individually adjusted based on the color of the clothes worn by the subject, which the color sensor discriminates.

### CROSS REFERENCE TO RELATED APPLICATIONS

This application relates to, but does not claim priority from, JP 2016-042081 filed Mar. 4, 2016 and published as JP 2017-153862 on Sep. 7, 2017, the entire contents of which are incorporated herein by reference.

### FIGURE SELECTED FOR PUBLICATION

FIG. 4.

### BACKGROUND OF THE INVENTION

### Field of the Invention

The present invention relates to a radiation imaging apparatus that comprises a radiation irradiation unit such as an X-ray tube and a radiation detector such as an X-ray detection detector that detects the radiation irradiated from the radiation irradiation unit and passes through the subject and carries out an X-ray fluoroscopy or an X-ray imaging.

### Description of the Related Art

With regard to the X-ray imaging apparatus as a radiation imaging apparatus, a collimator that is an aperture mechanism having a plurality of collimator-leaves, openable and closable, is installed in the irradiation region of the X-ray to regulate the X-ray exposure field that is an irradiation region of X-rays to the subject for the X-ray irradiated from the X-ray tube between the X-ray tube and the X-ray detector. When the X-ray exposure field relative to the subject to which the X-ray is irradiated from the X-ray tube is adjusted, by lighting a visible light, called a collimator lamp that is in-place in the conjugated location with the X-ray tube in the reverse side of the patient relative to the collimator, the irradiation exposure area of the visual light is formed and the X-ray exposure field, which is adjusted by the collimator mechanism, is confirmed by visually recognizing the irradiation field of the visible light.

Patent Document 1 discloses the portable X-ray apparatus comprising a modification means that modifies the light intensity to be irradiated to confirm the irradiation exposure filed without an impact of the brightness of the surrounding area and giving the subject discomfort using such a collimator.

In addition, Patent Document 2 discloses a collimator mechanism that is using the small LED, which has a long life and much less power consumption, as the light source and houses the LED inside the cone having a combined paraboloidal surface contractor shape.

In addition, Patent Document 3 discloses the X-ray imaging apparatus in which the operator can easily and certainly recognize the kind of selected added-filter by changing the color of the visible light forming the light exposure field by moving any one of multiple color filters according to the kind of selected added-filter.

### RELATED PRIOR ART DOCUMENTS

Patent Document 1—JP 2001-70292 A1
Patent Document 2—JP 2004-209259 A1
Patent Document 3—PCT/JP2011/050546—WO 2011/089686

### ASPECTS AND SUMMARY OF THE INVENTION

Objects to be Solved

Conventionally, when the collimator lamp is turned on to form the visible light exposure field and the color of the clothes of the subject and the color of the visible light have the similar color property, it is problematic that the edge (border) of the visible light exposure field is hard to recognize. For example, when the exam gown is reddish and the visible light from the collimator lamp is also reddish, it is hard to discriminate the edge of the visible light exposure field where the edge is in-place in case. In such a case, the operator must go to the subject side to make sure of the edge of the visible light exposure field or turn off the exam room light to make sure of the edge portion and so forth, for which the work is bothersome.

The purpose of the present invention is to solve the above objects and to provide a radiation imaging apparatus that facilitates and is feasible to discriminate the light exposure field of the visible light regardless of the color of the exam gown of the subject.

Means for solving the problem

According to the invention claimed in claims, an X-ray imaging apparatus comprises: a radiation irradiation unit; a radiation detector that detects a radiation that is irradiated from the radiation irradiation unit and transmits through a subject; a collimator that has a plurality of collimator-leaves that modifies an exposure field of the radiation that is irradiated from the radiation irradiation unit to the subject; a light source that emits a visible light to visually discriminate the radiation exposure field that is adjusted with the collimator-leaves, a discriminator that discriminates a color of clothes worn by the subject, and a color modification unit that modifies the color of the visible light emitted from the light source based on the color discriminated by the discriminator.

With regard to the radiation imaging apparatus according to the claimed invention set forth above, the discriminator is a color sensor that discriminates the color of clothes worn by the subject.

With regard to the radiation imaging apparatus according to another claimed invention set forth above, the light source further comprises a first LED that emits a red light, a second LED that emits a green light and a third LED that emits a blue light.

With regard to the radiation imaging apparatus according to another claimed invention set forth above, the light source further comprises a fourth LED that emits a white light.

With regard to the radiation imaging apparatus according to another claimed invention set forth above, the color modification unit individually adjusts each light amount of the first LED that emits the red light, the second LED that emits the green light and the third LED that emits the blue light based on the color of the clothes worn by the subject, which is discriminated by the color sensor.

With regard to the radiation imaging apparatus according to the claimed invention set forth above, the discriminator is an input unit to which the operation input the color of the clothes worn by the subject.

With regard to the radiation imaging apparatus according to another claimed invention set forth above, the color modification unit modifies the color of the visible light emitted from the light source, so that the color of the visible light emitted from the light source becomes equivalent to the complementary color of the color of the clothes worn by the subject.

### EFFECT OF THE INVENTION

According to the aspect of the invention claimed in claims, the color of the visible light emitted from the light source is modified based on the color of the clothes worn by the subject, so that the exposure field of the visible light is easily discriminated regardless the color of the clothes worn by the subject.

According to the aspect of the invention claimed in claims, the color sensor automatically discriminates the color of the clothes worn by the subject and the color of the visible light emitted from the light source is easily modified to the color by which the exposure field of the visible light is easily discriminated.

According to an aspect of the invention claimed in claims, the color of the visible light emitted from the light source is easily modified to the color, by which the exposure field of the visible light is easily discriminated, using the first LED that emits the red light, the second LED that emits the green light and the third LED that emits the blue light.

According to the aspect of the invention claimed in claims, the color of the visible light emitted from the light source is easily modified to the color by which the exposure field of the visible light is easily discriminated and in addition, the lighting (illumination) intensity of the light exposure field of the visible light is further intensified by the action of the LED that emits the white light.

According to the aspect of the invention claimed in claims, modification unit individually adjusts each amount of the first LED that emits the red light, the second LED that emits the green light and the third LED that emits the blue light based on the color of the clothes worn by the subject, which is discriminated by the color sensor.

According to the aspect of the invention claimed in claims, the color of the visible light emitted from the light source is easily modified by that the operator inputs.

According to the aspect of the invention claimed in claims, the color of the visible light emitted from the light source it modified to be equivalent to the complementary color of the color of the clothes worn the subject, so that the color of the visible light is automatically modified to the color by which the light exposure field is easily discriminated.

The above and other aspects, features and advantages of the present invention will become apparent from the following description read in conjunction with the accompanying drawings, in which like reference numerals designate the same elements.

### BRIEF DESCRIPTION OF THE DRAWINGS

FIG. 1 is a schematic view illustrating an X-ray imaging apparatus as a radiation imaging apparatus according to the present invention.

FIG. 2 is a schematic view illustrating the collimator 43 and so forth.

FIG. 3 is a schematic view illustrating the state in which the four collimator-leaves 53 forms an exposure field R of the X-ray.

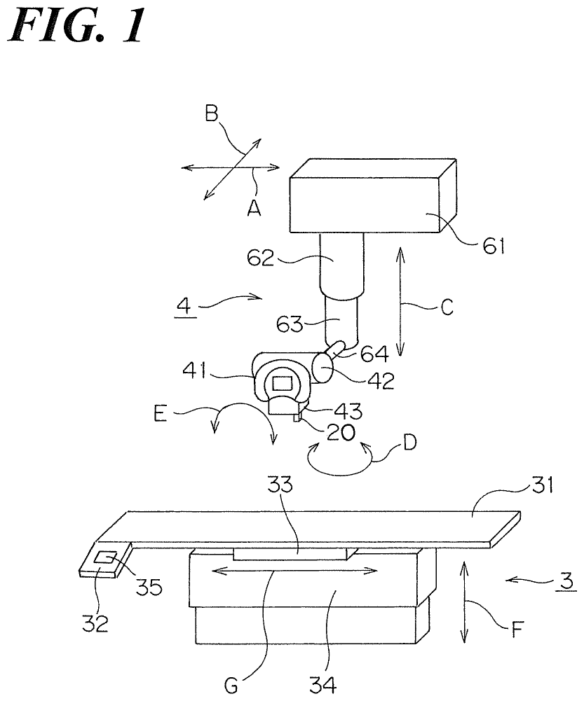

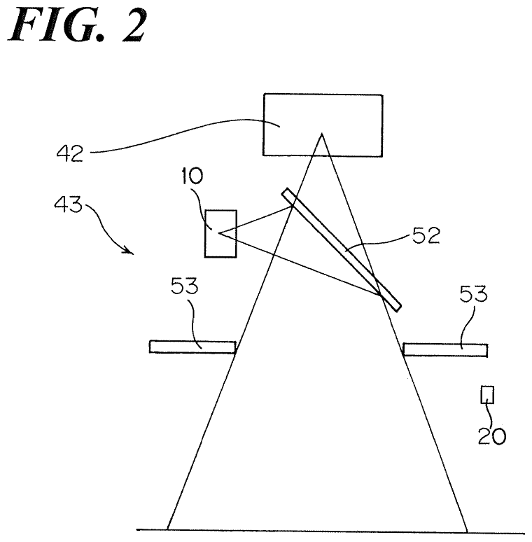

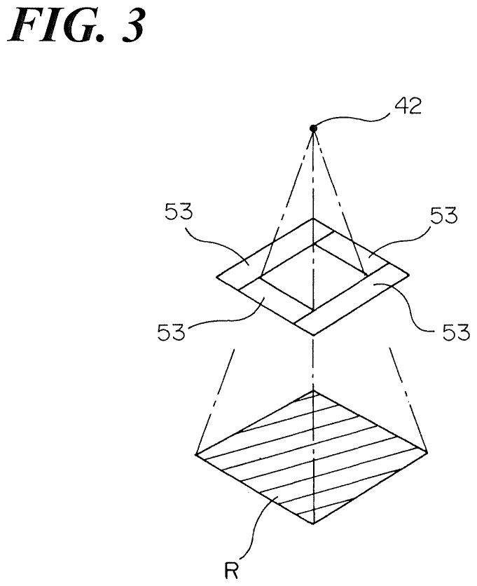

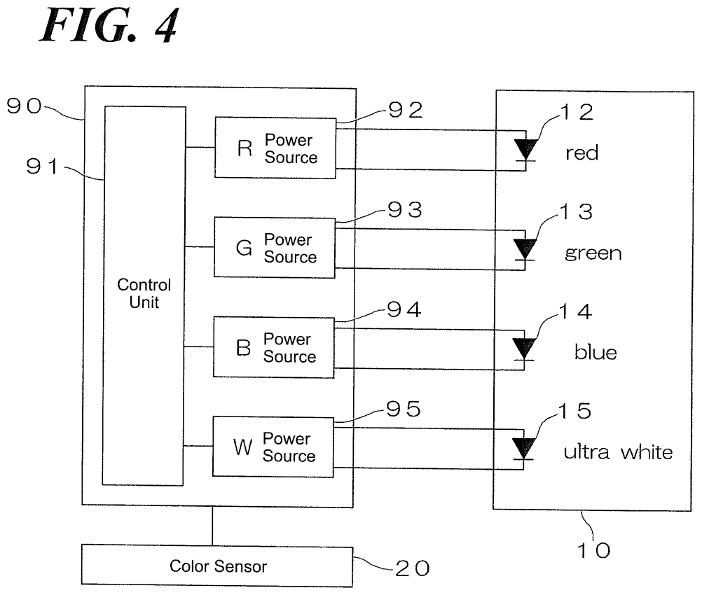

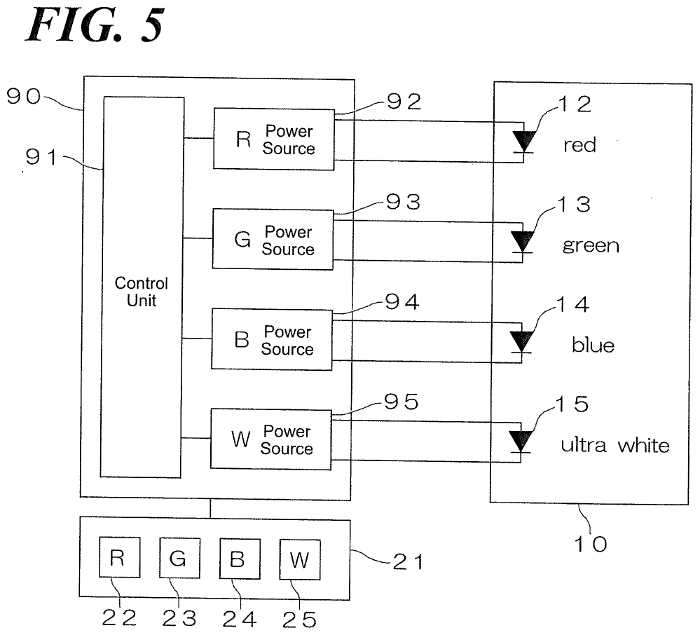

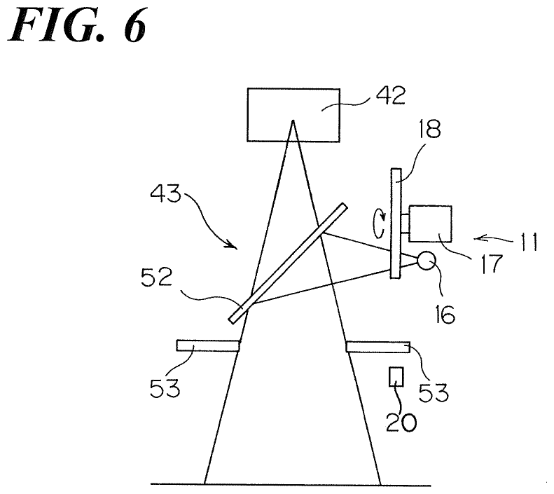

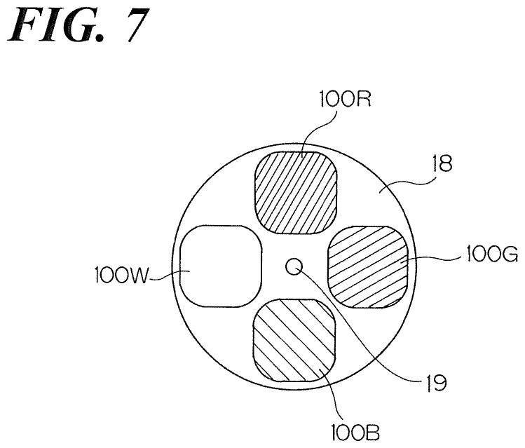

FIG. 4 is a block diagram illustrating the light source 10, the color modification unit 90 and the color sensor 20.

FIG. 5 is a block diagram illustrating the light source 10, the color modification unit 90 and the color switch 21.

FIG. 6 is a schematic view of the collimator 43 according to another Embodiment.

FIG. 7 is a plan view illustrating the rotation plate 18.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ or ‘connect’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

The inventors set forth Embodiments of the present invention based on the following FIGs. FIG. 1 is a schematic view illustrating an X-ray imaging apparatus as a radiation imaging apparatus according to the present invention.

The X-ray imaging apparatus comprises an X-ray imaging base 3 and an X-ray irradiation unit 4 that are installed in the X-ray imaging room. The X-ray imaging base 3 comprises a table 31 on which a subject is loaded, an operation panel 32 including a display 35 comprising a liquid crystal display with a touchscreen, a Bucky device 33 housing an X-ray detector such as a flat panel detector inside thereof, a lifting unit 34 that ups and downs the table 31 and the Bucky device 33. The Bucky device 33 is movable in the horizontal direction indicated as G-direction in FIG. 1. In addition, the Bucky device 33 ups and downs in the F-direction in FIG. 1 along with the table 31.

The X-ray irradiation unit 4 comprises: a support element 61 movable in the orthogonal directions, i.e., A-direction and B-direction, relative (parallel) to the ceiling of the on the X-ray imaging room; a hanging support column 62 extending downward from the support element 61; a shifting unit 63 telescopic in the C-direction and rotatable in the D-direction relative to the hanging support column 62; a supporting shaft 64 that is mounted on the bottom of the shifting unit 63 and rotates the operation unit 41, the X-ray tube 42, and the collimator 43 as a unit around in the E-direction. Accordingly, the X-ray tube 42 is movable together with the operation element 41 and the collimator 43 in the A-, B-, C-, D-, and E-direction. The color sensor 20, as set forth later, that discriminates the color of clothes worn by the subject is attached to the underside of the collimator 43.

FIG. 2 is a schematic view illustrating the collimator 43. In addition, FIG. 3 is the schematic view illustrating the state in which the four collimator-leaves 53 forms the exposure field R of the X-ray.

The collimator 43 comprises the four collimator-leaves 53 that restricts the exposure field R of the X-ray irradiated from the X-ray tube 42. In addition, the collimator 43 comprises the light source 10 that emits a visible light to visually discriminate the X-ray exposure field. The visible light emitted from the light source 10 is reflected toward the collimator-leaves 53 at the mirror 52, through which the X-ray is transmittable, and the rectangular light exposure field of the visible light is formed by the four collimator-leaves 53. The size of the exposure field of the visible light is identical to the size of the exposure field R of the X-ray. The operator can confirm the X-ray exposure field by confirming the exposure field of the visible light.

FIG. 4 is a block diagram illustrating the light source 10, the color modification unit 90 and the color sensor 20 that is the discriminator according to the present invention.

The color sensor 20 comprises and a digital color sensor including a plurality of photodiodes and is sensitive (detectable) to respective red (R), green (G) and blue (B) color. The color sensor 20 outputs the detection results of each RGB color as a digital value. Referring to FIG. 1, FIG. 2, the color sensor 20 is mounted to the underside of the collimator 43, so that the sensitivity thereof is maximum while facing the table 31 on which the subject is loaded.

In addition, the light source 10 further comprises a full color LED module including the first LED 12 that emits a red light, the second LED 13 that emits a green light, the third LED 14 that emits a blue light, and the fourth LED 15 that emits a white light.

In addition, the color modification unit 90 comprises the power source 92 for R connected to the first LED 12, the power source 93 for G connected to the second LED 13, the power source 94 for B connected to the third LED 14 and the power source 95 for W connected to the fourth LED 15. Such power sources 92, 93, 94, 95 adjusts individually the amount of the lights from the first LED 12, the second LED 13, the third LED 14, the fourth LED 15 by that the control unit 91 controls.

When the exposure field of the visible light is formed for confirming the X-ray exposure field relative to the subject using the X-ray imaging apparatus having such a structure, the color sensor 20 mounted to the underside of the collimator 43 detects the color of the clothes (gown) worn by the subject loaded on the table 31. Then, each light amount of the first LED 12 that emits the red light, the second LED 13 that emits the green light and the third LED 14 that emits the blue light and the fourth LED 15 that emits the white light is individually adjusted based on the color of the clothes worn by the subject, which the color sensor 20 discriminates.

At such a time, each light amount of the first LED 12 that emits the red light, the second LED 13 that emits the green light and the third LED 14 that emits the blue light and the fourth LED 15 that emits the white light is individually adjusted to provide an complementary color of the color of the gown worn by the subject corresponding to the color of the gown worn by the subject, so that the color of the visible light emitted from the light source 10 further facilitates the exposure field to be discriminable corresponding to the color of the clothes worn by the subject.

Further specifically, given the detection value of each RGB, which is detected by the color sensor 20, is respectively R1, G1, B1, each light amount R2 of the first LED 12, G2 of the second LED 13 and B2 of the third LED 14 is determined by the following formulae (see below). In addition, in such a case, the fourth LED 15 that emits the white light is set to provide a high-amount of the light to further increase the light intensity of the exposure field of the visible light. Accordingly, the color of the visible light emitted from the light source 10 is the color corresponding to the complementary color of the color of the gown worn by the subject, which is discriminated by the color sensor 20, and the color of the visible light forming the exposure field of the visible light automatically becomes the color facilitating the exposure field to be a discriminable color.

R2=(G1+B1)/2

G2=(B1+R1)/2

B2=(R1+G1)/2

In addition, according to the aspect of another Embodiment, only the LED, i.e., one of the first LED 12, the second LED 13, and the third LED 14, selected from a group consisting of the first LED 12, the second LED 13 and the fourth LED 14, which corresponds to the color having the highest detection value among the detection value R1 of R, the detection value G1 of G, and the detection value B1 of B, which the color sensor 20 respectively detects, is turned off. For example, given R1 among the respective detection values R1, G1 and B1 of each RGB detected by the color sensor 20 is the highest value, only the first LED 12 that emits the red light is turned off and, at the same time, the second LED 13, the third LED 14 and the fourth LED 15 are turned on.

For example, given the ratio between the detection value R1 of R, the detection value G1 of G and the detection value B1 of B detected by the color sensor 20 is 10/5/1, only the first LED 12 that emits the red light is turned off and, at the same time, the second LED 13, the third LED 14 and the fourth LED 15 are turned on.

In addition, according to the ratio between the detection value R1 of R, the detection value G1 of G and the detection value B1 of B detected by the color sensor 20, the amount of light of the respective first LED 12, second LED 13, third LED 14 is modified to correspond to the set-up values that are preset and stored in the memory using the table format.

Even in such a case, the color of the visible light emitted from the light source 10 is easily modified to the color by which the exposure field is easily discriminable. In such a case, the fourth LED 15 that emits the white light is turned on, so that the light intensity of the exposure field of the visible light is further intensified.

In addition, according to the aspect of the Embodiment set forth above, instead of the commercially available color sensor 20, a color recognition device comprising a camera taking the subject and an image processor that discriminates the color of the clothes worn by the subject by image-processing the image taken by such a camera can be applied to the color sensor of the present invention.

In addition, according to the aspect of the Embodiment set forth above, the fourth LED 15 that emits the white light is not mandatory. When the first LED 12 that emits the red light, the second LED 13 that emits the green light and the third LED 14 that emits the blue light that can secure the satisfactorily large amount of the light are used, the fourth LED 15 that emits the white light is not mandatory.

Next, the inventors set forth an alternative Embodiment of the present invention relative to the discriminator. FIG. 5 is a block diagram illustrating the light source 10, the color modification unit 90 and the color sensor 21 that is the discriminator according to the present invention. Further, the same member as illustrated in the Embodiment referring to FIG. 4 is not described in detail while providing the identical reference letter.

According to the aspect of the Embodiment referring to FIG. 4, a color sensor 20 as the discriminator of the present invention is applied to discriminate the color of the clothes worn by the subject. On the other hand, according to the aspect of the Embodiment referring to FIG. 5, a color switch 21 as the discriminator of the present invention by which the operator inputs the color of the clothes worn by the subject is applied thereto. Such a color switch 21 comprises a switch 22 that turns on the first LED 12 that emits the red light, a switch 23 that turns on the second LED 13 that emits the green light, a switch 24 that turns on the third LED 14 that emits the blue light and a switch 25 that turns on the fourth LED 15 that emits the white light.

When the exposure field of the visible light is formed for confirming the X-ray exposure field relative to the subject using the X-ray imaging apparatus according to the aspect of the Embodiment referring to FIG. 5, the operator checks out the color of the clothes (gown) worn by the subject loaded on the table 31. Then, the operator operates each switch 22, 23, 24 and 25 relative to the color switch 21, and creates the color of the visible light emitted from the light source 10, by which the exposure field is easily discriminable.

In addition, according to the aspect of the Embodiment referring to FIG. 5, the color switch 21 includes four switches 22, 23, 24 and 25, and such switches are operated, but the operator may modify the color of the visible light emitted from the light source 10 by changing the number of pressing-down relative to the single switch. In addition, instead of using such switches, an operation mechanism such as a lever can be applied. Further, instead of designating and modifying the color of the visible light emitted from the light source 10, the color of the visible light emitted from the light source 10 can be automatically modified by inputting the color of the clothes worn by the subject.

Next, the inventor sets forth an alternative Embodiment of the present invention relative to the collimator 43. FIG. 6 is a schematic view of the collimator 43 according to the alternative Embodiment. Further, the same member as illustrated in the Embodiment referring to FIG. 2 is not described in detail while providing the identical reference letter.

According to the aspect of the present Embodiment, the collimator 43 comprises; a rotation plate 18 mounting a plurality of different color filters and a motor 17 that rotates the rotation plate 18, and a light source 11 that selectively moves the one of the plurality of different color filters mounted to the rotation plate 18 from the white color light source 16 into the light path reaching to the subject.

FIG. 7 is the plan view illustrating the rotation plate 18.

The rotation plate 18 rotates around the axis 19 as the rotation center by driving the motor 17. The rotation plate 18 comprises four holes that are installed in the radial location from the axis 19. And, three holes out of four holes attaches each color filter 100R, 100G, 100B made of the transparent (light transmittable) plate having each different color to each other. And, the one last hole attaching no color filter is an opening 100W. Here, for example, the red light transmits the color filter 100R, the green light transmits the color filter 100G, and the blue light transmits the color filter 100B.

The light source 11 per se comprises a color modification unit. When the light source 11 is used, the rotation plate 18 rotates based on the directive from the color sensor 20 referring to FIG. 4 or the directive from the color switch 21 referring to FIG. 5, and the color filters 100R, 100G, 100B or the opening 100W moves selectively to the light path reaching the subject from the white light source 16 to provide the color of the exposure field of the visible light with the most easily discriminable color compared to the color of the clothes worn by the subject. Even when such a structure is adopted, the color of the visible light emitted from the light source 11 is easily modified to the color by which the exposure field of the visible light is easily discriminated.

### REFERENCE OF SIGNS

3 X-ray imaging base
4 X-ray irradiation unit
10 Light source
11 Light source
12 First LED
13 Second LED
14 Third LED
15 Fourth LED
16 White light source
17 Motor
18 Rotation plate
20 Color sensor
21 Color switch
31 Table
33 Bucky device
39 Leg
42 X-ray tube
43 Collimator
52 Mirror
53 Collimator-leaves

As used herein, a computing device broadly includes some form of an input device for receiving data, an output device for outputting data in tangible form (e.g. printing or transmitting data, or displaying on a computer screen), a memory for storing data as well as computer code, and a processor/microprocessor for executing computer code wherein said computer code resident in the memory will physically cause said processor/microprocessor to read-in data via said input device, process said data within said microprocessor and output said processed data via said output device.

It will be further understood by those of skill in the art that the apparatus and devices and the elements herein, without limitation, and including the sub components such as operational structures, circuits, communication pathways, and related elements, control elements of all kinds, display circuits and display systems and elements, any necessary driving elements, inputs, sensors, detectors, memory elements, processors and any combinations of these structures etc. as will be understood by those of skill in the art as also being identified as or capable of operating the systems and devices and subcomponents noted herein and structures that accomplish the functions without restrictive language or label requirements since those of skill in the art are well versed in related radiotherapy imaging devices, systems, and arrangements, including related radiotherapy tracking computers and operational controls and technologies of radiographic devices and all their sub components, including various circuits and components and combinations of circuits and combinations of components for such devices and for all related hand held type devices, without departing from the scope and spirit of the present invention.

Although only a few embodiments have been disclosed in detail above, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art.

Those of skill would further appreciate that the various illustrative logical blocks, modules, operating circuits, and algorithm steps described in connection with the embodiments disclosed herein may be implemented as electronic hardware, computer software running on a specific purpose machine that is programmed to carry out the operations described in this application, or combinations of both. To clearly illustrate this interchangeability of hardware and software, various illustrative components, blocks, modules, circuit illustrations, step-modes, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary embodiments.

The various illustrative logical blocks, modules, and circuits described in connection with the embodiments disclosed herein, may be implemented or performed with a general or specific purpose processor, or with hardware that carries out these functions, e.g., a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computer system that also has an internal bus connecting to cards or other hardware, running based on a system BIOS or equivalent that contains startup and boot software, system memory which provides temporary storage for an operating system, drivers for the hardware and for application programs, disk interface which provides an interface between internal storage device(s) and the other hardware, an external peripheral controller which interfaces to external devices such as a backup storage device, and a network that connects to a hard wired network cable such as Ethernet or may be a wireless connection such as a RF link running under a wireless protocol such as 802.11. Likewise, an external bus may be any of but not limited to hard wired external busses such as IEEE-1394 or USB. The computer system can also have a user interface port that communicates with a user interface, and which receives commands entered by a user, and a video output that produces its output via any kind of video output format, e.g., VGA, DVI, HDMI, display port, or any other form. This may include laptop or desktop computers, and may also include portable computers, including cell phones, tablets such as the IPAD™ and Android™ platform tablet, and all other kinds of computers and computing platforms.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein.

The steps of a method or algorithm described in connection with the embodiments disclosed herein may be embodied directly in hardware, in a software module executed by a processor, using cloud computing, or in combinations. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of tangible storage medium that stores tangible, non-transitory computer based instructions. An exemplary storage medium is coupled to the processor such that the processor can read information from, and write information to, the storage medium. In the alternative, the storage medium may be integral to the processor. The processor and the storage medium may reside in reconfigurable logic of any type.

Those of skill in the particular art will be recognized as having and having access to sophisticated radiotherapy tracking systems, circuits, and methods such that the skill level is high in science, technology, computers, programming, circuit design, and arrangement such that the described elements herein, after and following a review of this inventive disclosure and the inventive details herein, will be understood by those of skill in the art.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

The memory storage can also be rotating magnetic hard disk drives, optical disk drives, or flash memory based storage drives or other such solid state, magnetic, or optical storage devices. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media. The computer readable media can be an article comprising a machine-readable non-transitory tangible medium embodying information indicative of instructions that when performed by one or more machines result in computer implemented operations comprising the actions described throughout this specification.

Operations as described herein can be carried out on or over a web site. The website can be operated on a server computer, or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

The computers described herein may be any kind of computer, either general purpose, or some specific purpose computer such as a workstation. The programs may be written in C, or Java, Brew or any other programming language. The programs may be resident on a storage medium, e.g., magnetic or optical of any kind developed now or later developed e.g. the computer hard drive, a removable disk or media such as a memory stick or SD media, or other electronic recording medium. The programs may also be run locally, on a station, or over a an open or closed network without limitations thereto, for example, with a server or other machine sending signals to the local machine, which allows the local machine to carry out the operations described herein.

Also, the inventors intend that only those claims which use the words “means for” (specifically requiring the phrase “for” in “means for”) are intended to be interpreted under 35 USC 112 (f) paragraph. Moreover, no limitations from the specification are intended to be read into any claims, unless those limitations are expressly included in the claims.

It will be further understood that the method steps described herein shall be understood additionally as descriptive algorithms for the operation of the enclosed units, switches, modes, and devices and units to which they apply.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. A radiation imaging apparatus, comprising:
a radiation irradiation unit;
a radiation detector that detects a radiation that is irradiated from said radiation irradiation unit and transmits through a subject;
a collimator that comprises a plurality of collimator-leaves that modifies an exposure field of said radiation that is irradiated from said radiation irradiation unit to said subject;
a light source that emits a visible light to visually discriminate a radiation exposure field that is adjusted with said collimator-leaves;
a discriminator that discriminates a color of clothes worn by said subject; and
a color modification unit that modifies said color of said visible light, which said light source emits, based upon said color of a clothes worn by said subject, which said discriminator discriminates.

2. The radiation imaging apparatus, according to claim 1, wherein:
said discriminator is a color sensor that discriminates the color of clothes worn by said subject.

3. The radiation imaging apparatus, according to claim 2, wherein:
the light source further comprises a first LED that emits a red light, a second LED that emits a green light, and a third LED that emits a blue light.

4. The radiation imaging apparatus, according to claim 3, wherein:
said light source further comprises a fourth LED that emits a white light.

5. The radiation imaging apparatus, according to claim 3, wherein:
said color modification unit individually adjusts a light amount of each said first LED that emits said red light, said second LED that emits said green light and said third LED that emits said blue light based on said color of the clothes worn by said subject, which is discriminated by said color sensor.

6. The radiation imaging apparatus, according to claim 1, wherein:
said discriminator is an input unit to which an operation inputs said color of the clothes worn by said subject.

7. The radiation imaging apparatus. according to claim 1, wherein:
said color modification unit modifies said color of the visible light emitted from said light source, so that said color of the visible light emitted from said light source becomes an equivalent to a complementary color of said color of the clothes worn by said subject.
